(19) United States
(12) Patent Application Publication
(10) Pub. No.: US 2005/0226836 A1
(43) Pub. Date: Oct. 13, 2005

(54) Composition to permanently reshape the hair comprising at least one material modified by incorporating a metal

(76) Inventors: Aude Livoreil, Paris (FR); Gabin Vic, Venette (FR); Henri Samain, Bievres (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/094,641

(22) Filed: Mar. 31, 2005

Related U.S. Application Data
(60) Provisional application No. 60/572,105, filed on May 19, 2004.

(30) Foreign Application Priority Data
Mar. 31, 2004 (FR) 0450642
Sep. 1, 2004 (FR) 0409259

(51) Int. Cl. A61K 7/09

(52) U.S. Cl. 424/70.5

(57) ABSTRACT

The present disclosure relates to an aqueous reducing composition, for permanently reshaping the hair, comprising in a cosmetically acceptable medium, (i) at least one reducing agent, and (ii) at least one organic, inorganic or hybrid solid material which forms a three-dimensional repeating lattice capable of entrapping one or more metal ions or complexes and which has been modified by incorporating at least one oxidizing species chosen from transition metals used as salts, oxides or complexes with a ligand.

This application claims benefit of U.S. Provisional Application No. 60/572,105, filed May 19, 2004, and French Patent Application Nos. 04/50642, filed Mar. 31, 2004, and 04/09259, filed Sep. 1, 2004, all of which are hereby incorporated by reference.

The present disclosure relates to a reducing cosmetic composition to be used in a process to permanently reshape the hair, the composition comprising at least one reducing agent, and at least one material that has been modified by incorporating into it an oxidizing species. It also relates to a method for permanently reshaping the hair, which uses such modified materials.

To obtain a permanent reshaping of the hair, typically, the following process is employed: in a first step, the keratin disulfide bonds (cystine) of hair are opened with a composition comprising a reducing agent. The hair is optionally rinsed, and then the disulfide bonds are reformed in a second step by applying to the hair, which has been straightened or placed beforehand under tension with rollers or equivalent, an oxidizing composition, also called a fixing solution, so as to give the desired form to the hair. This method enables, for example, the waving of the hair, the uncurling, the backcombing or the straightening thereof.

The usual technologies for reshaping the hair, and especially the compositions to permanently reshape or straighten the hair that are based on thiol group containing products have good results with respect to the resulting shape. However, such compositions may also provide the hair with an unpleasant odor.

To compensate for this drawback, one solution has been to mask bad odors by adding a fragrance. However, this solution is not satisfactory as it does not actually remove these bad odors, but merely masks them.

Alternative solutions have been proposed. U.S. Pat. No. 5,184,630, for example, suggests the addition of a zeolite to a cosmetic composition, so as to reduce bad odors.

Oxidizing or fixing compositions which use manganese have also been proposed to act as a substitute for hydrogen peroxide, which is what is traditionally used to counteract the bad odors. Unfortunately, if successive permanent waving/coloring operations follow each other, these compositions may induce a perturbation in the color uptake.

Therefore, there is a need for new cosmetic compositions for use in a process to permanently reshape the hair, with the capability to reduce bad odors without inducing any perturbation in the color uptake during two or more successive waving/coloring operations.

After having carried out various studies, the present inventors have now quite surprisingly discovered that using a composition comprising zeolites which have been modified by incorporating therein a metal can result in a drastic reduction of bad odors without inducing any perturbation in the color uptake during successive permanent waving/coloring operations and can provide at the same time an intensive and long lasting curl, with also less damage to the hair in some cases, as compared to previously known compositions.

Thus, disclosed herein is an aqueous non coloring reducing composition for a permanent hair reshaping process, comprising, in a cosmetically acceptable medium:
(i) at least one reducing agent of formula (I):
   (H)_{m′}X(R)ₙ (I),
wherein
   X is P, S or SO₂,
   m′ is 0 or 1,
   n is 1, 2 or 3 and
   R is a linear or branched, saturated or unsaturated (C₁-C₂₀)hydrocarbon radical, optionally interrupted by a heteroatom, and optionally substituted by at least one moiety chosen from a hydroxy moiety, a halogen moiety, an amine moiety and a carboxy moiety, a ((C₁-C₃₀)alkoxy)carbonyl moiety, an amido moiety, a ((C₁-C₃₀)alkyl)amino carbonyl moiety, a (C₁-C₃₀)acyl)amino moiety, a mono or di((C₁-C₃₀)alkyl)amino moiety, a mono or dihydroxy((C₁-C₃₀)alkyl)amino moiety,
or any salt thereof, combined with a base, and
(ii) at least one organic, inorganic or hybrid solid material which forms a three-dimensional repeating lattice capable of entrapping at least one metal ion or at least one metal complex and which has been modified by incorporating therein at least one oxidizing species chosen from transition metals used as salts, oxides or complexes with a ligand.

In one embodiment, the solid material is a zeotype, such as a zeolite.

As used herein, ‘zeotypes’ is intended to refer to zeolite-type crystalline structures, in which all or part of silicon, aluminum or phosphorus atoms are replaced by one or more atoms from elements belonging to columns 2 to 13 of the periodic table, for example by atoms chosen from boron, gallium, iron, chromium, germanium, titanium, vanadium, manganese, cobalt, zinc, beryllium, copper, nickel, cobalt, molybdenum, ruthenium, rhenium, palladium and silver atoms.

A given zeotype may contain a plurality of substituting elements.

The modified zeolite is, in one embodiment, a natural or synthetic zeolite having the following formula (II):
XM′_{2/n}O.Al₂O₃.YSiO₂.ZH₂O (II)
wherein
M′ is a replaceable ion, such as a monovalent or divalent metal ion,
n is the valency of the M′ ion,
X is a metal oxide,
Y represents the number of silica molecules, and
Z represents the number of crystallization water molecules.

In one embodiment, M′ is Na, K, Ca, Mg or Ba.

The modified zeolite can, for example, be chosen from A, K, L, P-L, O, T, X, Y and Ω-type zeolites, zeolites with a high content of silica, sodalite, mordenite, analcime, crynoptyrolite, chabazite and erionite.

In one embodiment, the modified zeolite is a synthetic zeolite.

The modified material(s) defined above, such as zeolites, may be present in an amount ranging from 0.1 to 10%, for instance from 1 to 5% by weight, as compared to the total weight of the reducing composition.

The zeolite may be modified by incorporating a metal as a salt of formula:
M(S)ₙ (III),
wherein
M is a metal chosen from Ti, V, Cr, Mn, Fe, Co, Ni, Cu, Zn, Mo, Ru, Rh, Pd, and Ag;
S is an inorganic anion chosen from sulfate, sulfonate, phosphate, phosphonate, chloride, bromide and nitrate ions; or an organic anion selected from lactate, citrate, acetate ions, and
n ranges from 1 to 8.

Alternatively, the zeolite is modified by incorporating a metal as an oxide of formula:
M_{n′}(O)ₙ (IV),
wherein
M is a metal chosen from Ti, V, Cr, Mn, Fe, Co, Ni, Cu, Zn, Mo, Ru, Rh, Pd, and Ag;
n′ ranges from 1 to 5,
O is an oxygen atom and
n ranges from 1 to 8.

The zeolite may alternatively be modified by incorporating a metal as a complex with a ligand, the complex having the following formula:
M(L)ₙ (V),
wherein
M is a metal chosen from Ti, V, Cr, Mn, Fe, Co, Ni, Cu, Zn, Mo, Ru, Rh, Pd, and Ag;
L is an organic ligand chosen from cyclic and acyclic, saturated and unsaturated compounds, comprising carboxylate groups, primary, secondary or tertiary amines, hydroxy groups or an inorganic ligand selected from NH₃, and NO, and n ranges from 1 to 10.

In one embodiment, L may be an organic ligand chosen from pyridine, pyridazine, pyrimidine, pyrazine, imidazole, pyrazole, triazole, 2,2′-bispyridylamine, tris-(2-pyridylmethyl)amine, 1,4,7-triazacyclononane, 1,4,7-trimethyl-1,4,7-triazacyclononane, 1,5,9-trimethyl-1,5,9-triazacyclododecane, ((bis-(1-methylimidazol-2-yl)-methyl))-(2-pyridylmethyl)-amine, N,N′-((bis-(1-methylimidazol-2-yl)-methyl))-ethylenediamine, N-bis-(2-benzimidazolylmethyl)-aminoethanol, 2,6-bis-(bis-(2-benzimidazolylmethyl)aminomethyl)-4-methylphenol, N,N,N′,N′-tetrakis-(2-benzimidazolylmethyl)-2-hydroxy-1,3-diaminopropane, 2,6-bis(bis-(2-pyridylmethyl)aminomethyl)-4-methylphenol, 1,3-bis(bis-(2-benzimidazolylmethyl)aminomethyl)-benzol, sorbitol, mannitol, erythritol, adonitol, inositol, lactose, and optionally their salts.

In another embodiment, the organic ligand may be chosen from 1,4,8,11-tetraazacyclotetradecane, 1,8-diazabicyclo[5.4.0]undec-7-ene, ethylenediamine, nitrilotriacetic acid, tris(aminoethyl)amine, tris(aminomethyl)methane, 1,3,5-triaminocyclohexane, 2,6-dicarboxylic pyridine, pyridine-2,6-dicarboxylic acid, 2,2′,6′,2″-terpyridine, 1,10-phenanthroline, tris(2-pyridylmethyl)amine, tris(2-pyridylethyl)amine and N-carboxymethyl-N-(2-pyridylmethyl)glycine.

The reducing composition disclosed herein comprises at least one other known reducing agent of formula (I) such as thioglycolic acid or thiolactic acid and ester and amide derivatives thereof, for instance, glycerol monothioglycolate, cysteamine and its acylated (C₁-C₄)-derivatives such as N-acetyl-cysteamine or N-propionyl-cysteamine, cysteine, N-acetyl-cysteine, thiomalic acid, pantheteine, 2,3-dimercaptosuccinic acid, N-(mercaptoalkyl)-ω-hydroxyalkylamides such as those described in the patent application EP-A-354 835, N-mono or N,N-dialkylmercapto-4-butyramides such as those described in the patent application EP-A-368 763, aminomercaptoalkylamides such as those described in the patent application EP-A-432 000. N-(mercaptoalkyl)succinamic acid and N-(mercaptoalkyl)succinimide derivatives such as those described in the patent application EP-A-465342, alkylaminomercaptoalkylamides such as those described in the patent application EP-A-514 282, an azeotropic mixture of 2-hydroxypropyl thioglycolate and (2-hydroxy-1-methyl)ethyl thioglycolate described in French patent FR-A-2 679 448, mercaptoalkylaminoamides such as those described in French patent FR-A-2 692 481, N-mercaptoalkylalkanediamides described in the patent application EP-A-653 202, as well as formamidine sulfinic acid derivatives. These compounds may be used as salts if appropriate.

As used herein, the expression ‘non coloring’ is intended to define a composition containing neither a direct dye nor an oxidation dye that may give the fibers a visible-color in one application after a rinsing and a drying step. In other words, the color difference ΔE between a treated fiber and a not treated fiber is below 5 in the L*a*b* system, with ΔE=(ΔL*²+Δa*²+Δb*²)^{1/2}.

In one embodiment, the reducing composition according to the present disclosure also comprises at least one surfactant of non ionic, anionic, cationic or amphoteric type. Non-limiting examples thereof are the alkyl sulfates, alkyl benzene sulfates, alkyl ether sulfates, alkyl sulfonates, quaternary ammonium salts, alkyl betaines, oxyethylene alkyl phenols, fatty acid alkanolamides, oxyethylene fatty acid esters, as well as other non ionic hydroxypropylether surfactants.

When the reducing composition according to the present disclosure comprises at least one surfactant, it may be present in an amount up to 30% by weight, such as from 0.5 to 10% by weight, as compared to the total weight of the reducing composition.

In order to improve the cosmetic properties of the hair or to reduce or prevent damage to the hair, the reducing composition may also comprise a treating agent of cationic, anionic, non ionic or amphoteric nature.

Among the treating agents that may be used, there may be mentioned, as non-limiting examples, to those described in French patents FR 2 598 613 and FR 2 470 596. Volatile or non volatile, linear or cyclic silicones may also be used as treating agents, as well as mixtures thereof, polydimethylsiloxanes, quaternized polyorganosiloxanes such as those described in French Patent FR 2 535 730, polyorganosiloxanes with aminoalkyl moieties modified by alkoxycarbonylalkyl moieties such as those described in U.S. Pat. No. 4,749,732, polyorganosiloxanes such as polydimethylsiloxane-polyoxyalkyl copolymer i.e., dimethicone copolyol, polydimethylsiloxane with stearoxy end groups (stearoxy-dimethicone), polydimethylsiloxane and dialkylammonium acetate copolymer or polydimethylsiloxane and polyalkyl betaine copolymer described in French Patent No. FR 2 197 352, organo-polysiloxanes-modified by mercapto or mercaptoalkyl groups such as those described in French Patent No. FR 1 530 369 and in the European patent application EP 295 780, as well as silanes such as stearoxytrimethylsilane.

The reducing composition according to the present disclosure may also comprise at least one other treating ingredient such as cationic polymers such as those used in the compositions of French Patents Nos. FR 2 472 382 and FR 2 495 931, for example, cationic polymers such as of the ionene type, or cyclopolymers, basic aminoacids such as lysine or arginine, acidic aminoacids such as glutamic acid or aspartic acid, peptides and their derivatives, protein hydrolyzates or waxes.

The reducing composition according to the present disclosure may also comprise at least one swelling agent and/or penetrating agent, or at least one agent that is able to increase the reductor's efficiency, such as a SiO₂/PDMS mixture (polydimethylsiloxane), dimethylisosorbitol, urea and its derivatives, pyrrolidone, N-alkyl-pyrrolidohes, thiamorpholinone, alkyleneglycol or dialkyleneglycol alkyl ethers such as for example propyleneglycol monomethylether, dipropyleneglycol monomethylether, ethyleneglycol monoethylether and diethyleneglycol monoethylether, C₃-C₆ alkanediols such as for example 1,2-propanediol, 1,3-propanediol and 1,2-butanediol, 2-imidazolidinone, as well as other compounds such as fatty alcohols, lanoline derivatives, active agents such as panthothenic acid, agents for combating hair loss, anti-dandruff agents, thickeners, suspension agents, sequestering agents, opacifying agents, dyes, sunscreen agents as well as fragrances and preserving agents.

The reducing composition according to the present disclosure may also comprise a solvent such as, for example, ethanol, propanol, isopropanol or glycerol, to a maximum concentration of 20% by weight, as compared to the total weight of the composition.

The composition according to the present disclosure may, in one embodiment, comprise water or a combination of water and at least one C₁-C₆ lower alcohol, such as ethanol, isopropanol or t-butanol.

The reducing composition according to the present disclosure may, for example, be in substantially aqueous form, i.e., it comes as a lotion, thickened or not, as a cream, thickened or not, or as a gel.

When the compositions are to be used for a hair uncurling or straightening process, the reducing composition may, for instance, be in the form of a thickened cream, so as to maintain the hair as straight as possible. These creams are made in the form of “heavy” emulsions, for example glyceryl stearate, glycol stearate, auto-emulsifiable waxes or fatty alcohols-based emulsions.

Liquids or gels comprising certain thickeners may also be used such as carboxyvinyl polymers or copolymers, which “stick” and fix the hair in the smooth state during the exposure time.

The reducing composition according to the present disclosure can be an exothermic type composition as well, that is to say it causes a certain heating-up when applied onto the hair, which may be pleasant for the user who is experiencing the permanent waving or the uncurling first step.

The zeolites that have been modified by incorporating a metal or a metallic salt are generally prepared according to the procedure described below.

A zeolite modified according to the present disclosure is prepared by contacting the zeolite with an aqueous solution comprising a metal ion. This contact causes the zeolite exchangeable ion to be replaced by the aqueous solution ion. This contacting step may be carried out at a temperature ranging from 10 to 70° C., such as from 40° C. to 60° C., for 10 to 24 hours. The aqueous solution pH is adjusted to between 5 and 7. This adjustment prevents the metal oxides from precipitating on the zeolite surface. The metal ions in the aqueous solution are, in one embodiment, introduced as a metallic salt. As an example, silver ions used may be in the form of silver nitrate, silver sulfate or silver acetate.

The metal content of the zeolite may be easily adjusted by those skilled in the art by modifying the metal concentration in the aqueous solution.

A modified zeolite suitable for carrying out the invention is, for example, the zeolite marketed by ATO-CECA-DAFA under the trade name ZEOSTOP X™.

Also disclosed herein is an aqueous reducing composition, comprising at least one reducing agent and at least one solid organic material as a zeotype.

The solid material is, for example, a zeotype comprising an oxidizing species which is an integral part of its three-dimensional lattice. The oxidizing species may, in one embodiment, be a metal chosen from Ti, V, Cr, Mn, Fe, Co, Ni, Cu, Zn, Mo, Ru, Rh, Pd, and Ag.

The metal may come as a complex with a ligand, the complex having formula:
M(L)ₙ (V),
wherein
M is a metal chosen from Ti, V, Cr, Mn, Fe, Co, Ni, Cu, Zn, Mo, Ru, Rh, Pd, and Ag;
L is an organic ligand chosen from cyclic and acyclic, saturated and unsaturated compounds, comprising carboxylate groups, primary, secondary or tertiary amines, hydroxy groups or an inorganic ligand selected from NH₃, and NO, and
n ranges from 1 to 10.

The organic ligand may, for example, be selected from: 1,4,8,11-tetraazacyclotetradecane, 1,8-diazabicyclo[5.4.0]undec-7-ene, ethylenediamine, nitrilotriacetic acid, tris(aminoethyl)amine, tris(aminomethyl)methane, 1,3,5-triaminocyclohexane, 2,6-dicarboxylic pyridine, pyridine-2,6-dicarboxylic acid, 2,2′,6′,2″-terpyridine, 1,10-phenanthroline, tris(2-pyridylmethyl)amine, tris(2-pyridylethyl)amine, and N-carboxymethyl-N-(2-pyridylmethyl)glycine.

Methods for preparing titanium and iron based zeotypes suitable to carry out the present disclosure are respectively described in U.S. Pat. Nos. 4,480,135 and 5,110,995.

U.S. Pat. No. 4,480,135, for example, describes how to prepare a titanium silicalite (TS-1) from a reaction mixture comprising a silica oxide, a titanium oxide, optionally an alkaline oxide, a nitrogen organic base and water. The silica oxide may derive from a tetraalkylorthosilicate, a colloidal silicate or an alkali metal silicate, as for example Na, or K.

The titanium oxide derives from a hydrolysable compound for example TiCl₄ or TiOCl₂ Ti(alkoxy)₄; such as Ti(OC₂H₅)₄. The organic base may be tetraalkylammonium hydroxide, such as tetrapropylammonium hydroxide.

The reaction mixture undergoes a hydrothermal treatment in an autoclave, at a temperature ranging from 130° C. to 200° C., under its own pressure, for 6 to 30 days, until TS-1 precursor crystals develop. Crystals are separated from the stock solution, cautiously rinsed out and dried. In the anhydrous state, the composition of the crystals is as follows:
xTiO₂(1-x)SiO₂.0.04(RN+)₂O
where x ranges from 0.0001 to 0.04.

The precursor crystals are heated in the air for 1 to 72 hours at a temperature of 550° C., so as to completely remove the nitrogen organic base. The composition of the resulting titanium silicalite is as follows:
xTiO₂.(1-x)SiO₂

U.S. Pat. No. 5,110,995 describes an iron based zeotype preparation from a mixture comprising a silica source, an iron source, and if necessary, a EIⁿ⁺ source (where EI is an element belonging to the periods 2, 3, 4 or 5 of the periodic table of the elements), a base, an organic surfactant and optionally a seed crystal.

The reaction mixture undergoes a hydrothermal treatment in an autoclave, for 1 to 30 days, at a temperature ranging from 80° C. to 200° C. Once crystallization is complete, crystals are filtered out, washed with distilled water and then dried. The product is finally calcined at a temperature ranging from 520° C. to 550° C., so as to remove the organic inclusions. If necessary, a decationization may be effected with a solution of NH₄OH+NH₄Cl or with a solution made of inorganic acids. If necessary, an element may be introduced in the Fe—Si matrix by means of an ion exchange method or by impregnation.

Also disclosed herein is a method for permanently reshaping the hair using a reducing composition comprising at least one reducing agent and a modified material.

The permanent hair reshaping method according to the present disclosure comprises:
applying to the hair a reducing composition as described above, to reduce the keratin disulfide bonds;
re-forming the disulfide bonds by applying to the hair an oxidizing composition or by contacting the hair with atmospheric oxygen (an oxidizing-fixing step).

The modified material may, for example, be incorporated into the reducing composition when it is ready for use.

Alternatively, the modified material is applied onto the hair before applying a reducing composition. Accordingly, the present disclosure also provides for a method for permanently reshaping the hair comprising:
applying to the hair an aqueous composition comprising, in a cosmetically acceptable medium, at least one organic, inorganic or hybrid solid material which forms a three-dimensional repeating lattice capable of entrapping at least one metal ion or at least one metal complex and which has been modified by incorporating at least one oxidizing species chosen from transition metals, used as salts, oxides or complexes with a ligand;
applying to the hair an aqueous reducing composition to reduce the keratin disulfide bonds, the composition comprising, in a cosmetically acceptable medium, at least one reducing agent of formula:
   (H)_{m′}X(R)ₙ (I),
   wherein
   X is P, S or SO₂,
   m′ is 0 or 1,
   n is 1, 2 or 3 and
   R is a linear or branched, saturated or unsaturated (C₁-C₂₀)hydrocarbon radical, optionally interrupted by a heteroatom, and optionally substituted by at least moiety chosen from a hydroxy moiety, a halogen moiety, an amine moiety and a carboxy moiety, a ((C₁-C₃₀)alkoxy)carbonyl moiety, an amido moiety, a ((C₁-C₃₀)alkyl)amino carbonyl moiety, a (C₁-C₃₀)acyl)amino moiety, a mono or di((C₁-C₃₀)alkyl)amino moiety, a mono and dihydroxy((C₁-C₃₀)alkyl)amino moiety, and any salt thereof, combined with a base, and
re-forming the disulfide bonds by applying to the hair an oxidizing composition or by contacting the hair with atmospheric oxygen (an oxidizing-fixing step).

It is possible that the hair is set in form using mechanical means well known to those skilled in the art.

To obtain a permanent waving of the hair, rollers, for example, may be used, the reducing composition being applied before or after the hair setting process and the oxidizing-fixing composition being applied after the reducing composition, with or without an intermediate or a subsequent step to rinse out or to apply an intermediate composition.

The reducing composition according to the present disclosure is, in one embodiment, applied to wet hair, which has been previously wrapped round rollers of 2 to 30 mm diameter. The composition may also be applied as the hair rolling process progresses. The reducing composition is then left for a period of time ranging from 5 to 60 minutes, such as from 15 to 45 minutes to act, then has to be rinsed out thoroughly. The oxidizing composition is then applied to wrapped hair, the oxidizing composition enabling the re-formation of the keratin disulfide bonds, for an application time ranging from 2 to 10 minutes. After the rollers are removed, the hair is rinsed out thoroughly.

Once the reducing composition has been applied, the hair may also undergo a thermal treatment by heating at a temperature ranging from 30 to 60° C. for all or part of the application time. In practice, this step may be conducted using a helmet hair drier, a hair drier, an infrared radiation drier or other usual heating devices.

A heating iron may also be used, the temperature of which varies from 60 to 250° C., such as from 120 to 220° C., both as a heating process and as a hair setting process, the heating iron being used between the application step of the reducing composition and the fixing step.

To carry out the uncurling or the straightening of the hair, a reducing composition according to the present disclosure has to be applied onto the hair, then the hair undergoes a mechanical reshaping, so as to get fixed in its new shape by a smoothing operation with wide toothed comb, with back of the comb or back of the hand. After generally 5 to 60 minutes application time, such as 15 to 45 minutes, the hair has to be smoothed down once again, then carefully rinsed out, and the oxidizing composition is applied such as defined above, and has to be left on for a time ranging from 2 to 10 minutes before the hair is rinsed thoroughly.

The oxidizing composition may be any oxidizing composition traditionally used to permanently reshape hair and which contains an oxidizing agent selected, for instance, from hydrogen peroxide, alkaline bromates, persalts, polythionates and a mixture made of alkaline bromate and persalt. Hydrogen peroxide concentration may range from 1 to 20 volumes, such as from 1 to 10 volumes, alkaline bromate concentration may range from 2 to 12% and persalt concentration from 0.1 to 15% by weight, as compared to the total weight of the oxidizing composition.

The oxidizing composition pH may range from 2 to 10.

As explained before, the oxidizing composition may be applied immediately after the reducing composition or later.

According to one embodiment of the presently disclosed method, the smoothing of the hair may also be done, in all or part, with a heating iron at a temperature ranging from 60 to 250° C., such as from 120 to 220° C.

Also disclosed herein is a kit for permanently reshaping the hair, comprising, in a first compartment, a reducing composition according to the present disclosure comprising at least one compound of formula (I) and/or (II) as defined above and/or at least one organic or mineral salt thereof, and in a second compartment, an oxidizing composition.

The oxidizing composition may comprise at least one oxidizing agent chosen from hydrogen peroxide, alkaline bromates, persalts, polythionates and a mixture of alkaline bromate and persalt.

The present disclosure also relates to the use of an organic, inorganic or hybrid solid material which forms a three-dimensional repeating lattice capable of entrapping at least one metal ion or at least one metal complex, and which has been modified by incorporating at least one oxidizing species chosen from transition metals, used as salts, oxides or complexes with a ligand, to reduce the bad odor problem which may be inherent in methods for permanently reshaping the hair.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contains certain errors necessarily resulting from the standard deviation found in their respective testing measurements.

The following examples are intended to illustrate the invention in a non-limiting manner.

### EXAMPLE 1

### Composition Example

As a “control”, a reducing composition to permanently reshape hair according to the present disclosure was prepared by mixing the following compounds together:

A1

| | |
|---|---|
| Thioglycolic acid: | 9 g |
| 20% ammonia: | qs pH 9 |
| Demineralized water: | qs 100 g |

A reducing composition to permanently reshape hair according to the present disclosure was also prepared by mixing the following compounds together:

A2

| | |
|---|---|
| Thioglycolic acid: | 9 g |
| ZEOSTOP X₍₁₎: | 5 g |
| 20% ammonia: | qs pH 9 |
| Demineralized water: | qs 100 g |

₍₁₎ZEOSTOP X zeolite, marketed by CECA, comprising Cu, ZN, and Ag.

Finally, the following oxidizing composition was prepared:

B

| | |
|---|---|
| Hydrogen peroxide: | 8 vol. |
| Citric acid: | qs pH 3 |
| Demineralized water: | qs 100 g |

After being washed, the wet hair was rolled up on set rollers. The reducing composition A1 or A2 was applied onto the hair and left on for about 15 minutes, at ambient temperature, under a plastic film. The plastic film was removed and the odor evaluated. The composition was then rinsed out with water and the fixing composition B was applied, and left on for 5 minutes at ambient temperature.

The composition was rinsed out, rollers were removed and the hair curling was evaluated. For hair treated with the composition A1, then with the composition B, the bad odor was much stronger than for the hair treated with the composition A2 and then the composition B, for a similar resulting curling.

1. An aqueous non coloring reducing composition, for permanently reshaping the hair, comprising, in a cosmetically acceptable medium:
(i) at least one reducing agent of formula:
(H)_{m′}X(R)ₙ (I),
wherein
X is P, S or SO₂,
m′ is 0 or 1,
n is 1, 2 or 3 and
R is a linear or branched, saturated or unsaturated (C₁-C₂₀)hydrocarbon radical, optionally interrupted by a heteroatom, optionally substituted by at least one moiety chosen from a hydroxy moiety, a halogen moiety, an amine moiety and a carboxy moiety, a ((C₁-C₃₀)alkoxy)carbonyl moiety, an amido moiety, a ((C₁-C₃₀)alkyl)amino carbonyl moiety, a (C₁-C₃₀)acyl)amino moiety, a mono and di((C₁-C₃₀)alkyl)amino moiety, a mono and dihydroxy((C₁-C₃₀)alkyl)amino moiety,
or any salt thereof, combined with a base, and
(ii) at least one organic, inorganic or hybrid solid material which forms a three-dimensional repeating lattice capable of entrapping at least one metal ion or at least one metal complex and which has been modified by incorporating therein at least one oxidizing species chosen from transition metals used as salts, oxides or complexes with a ligand.

2. A composition according to claim 1, wherein the modified solid material is a zeolite.

3. A composition according to claim 2, wherein the modified zeolite is a natural or a synthetic zeolite having the formula:
XM′_{2/n}O.Al₂O₃.YSiO₂.ZH₂O (II)
wherein
M′ is a replaceable ion,
n is the valency of the M′ ion,
X is a metal oxide,
Y represents the number of silica molecules, and
Z represents the number of crystallization water molecules.

4. A composition according to claim 2, wherein the modified zeolite is chosen from A, K, L, P-L, O, T, X, Y and Ω-type zeolites, zeolites with a high content of silica, sodalite, mordenite, analcime, crynoptyrolite, chabazite and erionite.

5. A composition according to claim 2, wherein the modified zeolite is a synthetic zeolite.

6. A composition according to claim 2, wherein M′ is chosen from Na, K, Ca, Mg and Ba.

7. A composition according to claim 2, wherein the zeolite is modified by incorporating a metal as a salt of formula:
M(S)ₙ (III),
wherein
M is a metal chosen from Ti, V, Cr, Mn, Fe, Co, Ni, Cu, Zn, Mo, Ru, Rh, Pd, and Ag;
S is an inorganic anion chosen from sulfate sulfonate, phosphate, phosphonate, chloride, bromide, and nitrate ions; or an organic anion selected from lactate, citrate and acetate ions, and
n ranges from 1 to 8.

8. A composition according to claim 2, wherein the zeolite is modified by incorporating a metal as an oxide of formula:
M_{n′}(O)ₙ (IV),
wherein
M is a metal chosen from Ti, V, Cr, Mn, Fe, Co, Ni, Cu, Zn, Mo, Ru, Rh, Pd, and Ag;
n′ ranges from 1 to 5,
O is an oxygen atom and
n ranges from 1 to 8.

9. A composition according to claim 2, wherein the zeolite is modified by incorporating a metal as a complex with a ligand, said complex having formula:
M(L)ₙ (V),
wherein
M is a metal chosen from Ti, V, Cr, Mn, Fe, Co, Ni, Cu, Zn, Mo, Ru, Rh, Pd, and Ag;
L is an organic ligand chosen from cyclic and acyclic, saturated and unsaturated compounds, comprising carboxylate groups, primary, secondary or tertiary amines, hydroxy groups, or an inorganic ligand chosen from NH₃, and NO, and n ranges from 1 to 10.

10. A composition according to claim 9, wherein the organic ligand is chosen from 1,4,8,11-tetraazacyclotetradecane, 1,8-diazabicyclo[5.4.0]undec-7-ene, ethylenediamine, nitrilotriacetic acid, tris(aminoethyl)amine, tris(aminomethyl)methane, 1,3,5-triaminocyclohexane, 2,6-dicarboxylic pyridine, pyridine-2,6-dicarboxylic acid, 2,2′,6′,2″-terpyridine, 1,10-phenanthroline, tris(2-pyridylmethyl)amine, tris(2-pyridylethyl)amine, and N-carboxymethyl-N-(2-pyridylmethyl)glycine.

11. A composition according to claim 1, wherein the at least one solid material is a zeotype comprising an oxidizing species which is an integral part of its three-dimensional lattice.

12. A composition according to claim 11, wherein the oxidizing species is a metal chosen from Ti, V, Cr, Mn, Fe, Co, Ni, Cu, Zn, Mo, Ru, Rh, Pd, and Ag.

13. A composition according to claim 12, wherein the metal is in the form of a complex in combination with a ligand, said complex having formula:
M(L)ₙ (V),
wherein
M is a metal chosen from Ti, V, Cr, Mn, Fe, Co, Ni, Cu, Zn, Mo, Ru, Rh, Pd, and Ag;
L is an organic ligand chosen from cyclic and acyclic, saturated and unsaturated compounds, comprising carboxylate groups, primary, secondary or tertiary amines, hydroxy groups, or an inorganic ligand selected from NH₃, and NO, and n ranges from 1 to 10.

14. A composition according to claim 13, wherein the organic ligand is chosen from 1,4,8,11-tetraazacyclotetradecane, 1,8-diazabicyclo[5.4.0]undec-7-ene, ethylenediamine, nitrilotriacetic acid, tris(aminoethyl)amine, tris(aminomethyl)methane, 1,3,5-triaminocyclohexane, 2,6-dicarboxylic pyridine, pyridine-2,6-dicarboxylic acid, 2,2′,6′,2″-terpyridine, 1,10-phenanthroline, tris(2-pyridylmethyl)amine, tris(2-pyridylethyl)amine and N-carboxymethyl-N-(2-pyridylmethyl)glycine.

15. A composition according to claim 1, wherein the at least one solid material is present in an amount ranging from 0.1 to 10%, relative to the total weight of the composition.

16. A composition according to claim 15, wherein the at least one solid material is present in an amount ranging from 1 to 5%, relative to the total weight of the composition.

17. A composition according to claim 1, wherein the at least one reducing agent is chosen from thioglycolic acid, thiolactic acid, glycerol monothioglycolate, cysteamine, N-acetyl-cysteamine, N-propionyl-cysteamine, cysteine, N-acetyl-cysteine, thiomalic acid, pantheteine, 2,3-dimercaptosuccinic acid, N-(mercaptoalkyl)-ω-hydroxyalkylamides, N-mono or N,N-dialkylmercapto-4-butyramides, aminomercapto-alkylamides, N-(mercaptoalkyl)succinamic acid derivates and N-(mercaptoalkyl)succinimides derivates, alkylamino mercaptoalkylamides, an azeotropic mixture of 2-hydroxypropyle thioglyconate and (2-hydroxy-1-methyl)ethyl thioglycolate, mercaptoalkylaminoamides, N-mercapto-alkylalcanediamides and formamidine sulfinic acid derivates, and their salts.

18. A composition according to claim 1, further comprising at least one surfactant chosen from alkyl sulfates, alkyl benzene sulfates, alkyl ether sulfates, alkyl sulfonates, quaternary ammonium salts, alkyl betaines, oxyethylene alkyl phenols, fatty acid alkanolamides, oxyethylene fatty acid esters and hydroxypropylethers.

19. A composition according to claim 18, wherein the at least one surfactant is present in an amount up to 30% by weight, relative to the total weight of the composition.

20. A composition according to claim 19, wherein the at least one surfactant is present in an amount ranging from 0.5 to 10% by weight, relative to the total weight of the composition.

21. A composition according to claim 1, further comprising water or a mixture of water and at least one C₁-C₆ lower alcohol.

22. A composition according to claim 1, wherein the at least one C₁-C₆ lower alcohol is chosen from ethanol, isopropanol and t-butanol.

23. A composition according to claim 1, wherein the composition is in substantially aqueous form.

24. A composition according to claim 23, wherein the composition is in the form of an optionally thickened lotion an optionally thickened cream, or a gel.

25. A process for permanently reshaping hair comprising:
applying to the hair a reducing composition to reduce the keratin disulfide bonds; wherein said reducing composition comprises, in a cosmetically acceptable medium:
(i) at least one reducing agent of formula:
(H)_{m′}X(R)ₙ (I),
wherein
X is P, S or SO₂,
m′ is 0 or 1,
n is 1, 2 or 3 and
R is a linear or branched, saturated or unsaturated (C₁-C₂₀)hydrocarbon radical, optionally interrupted by a heteroatom, optionally substituted by at least one moiety chosen from a hydroxy moiety, a halogen moiety, an amine moiety and a carboxy moiety, a ((C₁-C₃₀)alkoxy)carbonyl moiety, an amido moiety, a ((C₁-C₃₀)alkyl)amino carbonyl moiety, a (C₁-C₃₀)acyl)amino moiety, a mono and di((C₁-C₃₀)alkyl)amino moiety, a mono and dihydroxy((C₁-C₃₀)alkyl)amino moiety,
or any salt thereof, combined with a base, and
(ii) at least one organic, inorganic or hybrid solid material which forms a three-dimensional repeating lattice capable of entrapping at least one metal ion or at least one metal complex and which has been modified by incorporating therein at least one oxidizing species chosen from transition metals used as salts, oxides or complexes with a ligand,
re-forming said disulfide bonds by applying to the hair an oxidizing composition or by contacting the hair with atmospheric oxygen.

26. A process according to claim 25, wherein the at least one modified material is incorporated into the reducing composition as it is being used.

27. A process for permanently reshaping the hair comprising:
applying to the hair an aqueous composition comprising, in a cosmetically acceptable medium, at least one organic, inorganic or hybrid solid material which forms a three-dimensional repeating lattice capable of entrapping at least one metal ion or at least one metal complex and which has been modified by incorporating at least one oxidizing species chosen from transition metals, used as salts, oxides or complexes with a ligand;
applying to the hair an aqueous reducing composition, to reduce the keratin disulfide bonds comprising, in a cosmetically acceptable medium, at least one reducing agent of formula:
(H)_{m′}X(R)ₙ (I),
wherein
X is P, S or SO₂,
m′ is 0 or 1,
n is 1, 2 or 3 and
R is a linear or branched, saturated or unsaturated (C₁-C₂₀)hydrocarbon radical, optionally interrupted by a heteroatom, optionally substituted by at least one moiety chosen from a hydroxy moiety, a halogen moiety, an amine moiety and a carboxy moiety, a ((C₁-C₃₀)alkoxy)carbonyl moiety, an amido moiety, a ((C₁-C₃₀)alkyl)amino carbonyl moiety, a (C₁-C₃₀)acyl)amino moiety, a mono and di((C₁-C₃₀)alkyl)amino moiety, a mono and dihydroxy((C₁-C₃₀)alkyl)amino moiety,
or any salt thereof, combined with a base, and
re-forming said disulfide bonds by applying to the hair an oxidizing composition or by contacting the hair with atmospheric oxygen.

28. A process according to claim 25, wherein the at least one reducing composition is left on the hair for 5 to 60 minutes.

29. A process according to claim 28, wherein the at least one reducing composition is left on the hair for 15 to 45 minutes.

30. A process according to claim 27, wherein the at least one reducing composition is left on the hair for 5 to 60 minutes.

31. A process according to claim 30, wherein the at least one reducing composition is left on the hair for 15 to 45 minutes.

32. A process according to claim 25, wherein the oxidizing composition comprises at least one oxidizing agent chosen from hydrogen peroxide, alkaline bromates, persalts, polythionates and a mixture of alkaline bromate and persalt.

33. A process according to claim 27, wherein the oxidizing composition comprises at least one oxidizing agent chosen from hydrogen peroxide, alkaline bromates, persalts, polythionates and a mixture of alkaline bromate and persalt.

34. A process according to claim 25, comprising heating the hair with a heating iron at a temperature ranging from 60 to 250° C., after applying the reducing composition to the hair and before applying the oxidizing composition.

35. A process according to claim 34, wherein said temperature ranges from 120 to 220° C.

36. A process according to claim 27, comprising heating the hair with a heating iron at a temperature ranging from 60 to 250° C., after applying the reducing composition to the hair and before applying the oxidizing composition.

37. A process according to claim 36, wherein said temperature ranges from 120 to 220° C.

38. A kit for permanently reshaping the hair, comprising
at least one first compartment comprising at least one reducing composition comprising, in a cosmetically acceptable medium:
(ii) at least one reducing agent of formula:
(H)_{m′}X(R)ₙ (I),
wherein
X is P, S or SO₂,
m′ is 0 or 1,
n is 1, 2 or 3 and
R is a linear or branched, saturated or unsaturated (C₁-C₂₀)hydrocarbon radical, optionally interrupted by a heteroatom, optionally substituted by at least one moiety chosen from a hydroxy moiety, a halogen moiety, an amine moiety and a carboxy moiety, a ((C₁-C₃₀)alkoxy)carbonyl moiety, an amido moiety, a ((C₁-C₃₀)alkyl)amino carbonyl moiety, a (C₁-C₃₀)acyl)amino moiety, a mono and di((C₁-C₃₀)alkyl)amino moiety, a mono and dihydroxy((C₁-C₃₀)alkyl)amino moiety,
or any salt thereof, combined with a base, and
(ii) at least one organic, inorganic or hybrid solid material which forms a three-dimensional repeating lattice capable of entrapping at least one metal ion or at least one metal complex and which has been modified by incorporating therein at least one oxidizing species chosen from transition metals used as salts, oxides or complexes with a ligand, and
at least one second compartment comprising an oxidizing composition.
